(19) United States
(12) Patent Application Publication
(10) Pub. No.: US 2026/0321410 A1
(43) Pub. Date: Oct. 15, 2026

(54) DETERMINATION OF A STATE OF A COMMON RESOURCE

(71) Applicant: Maquet Critical Care AB, Solna (SE)

(72) Inventor: Thomas WETTERBERG, Järfälla (SE)

(21) Appl. No.: 19/679,194

(22) Filed: May 15, 2026

Related U.S. Application Data
(63) Continuation of application No. PCT/EP2024/082261, filed on Nov. 13, 2024.

(30) Foreign Application Priority Data
Nov. 17, 2023 (SE) 2351313-8

(51) Int. Cl. G16H 40/40; G16H 40/67

(52) U.S. Cl. CPC G16H 40/40; G16H 40/67

(57) ABSTRACT

The present disclosure relates to a system comprising a plurality of medical devices and a common resource supplying the medical devices with a flow of medical gas. In particular, a method is disclosed, in which sensor data indicating a characteristic of the flow of a medical gas is received from each of the plurality of medical devices, a performance measure indicating a performance of the common resource is determined based on the sensor data, the performance measure is compared with a reference measure, and a state of the common resource is determined based on the comparison.

### CROSS-REFERENCE TO RELATED APPLICATIONS

This application is a continuation under 35 U.S.C. § 120 International Application No. PCT/EP2024/082261, filed Nov. 13, 2014, which claims priority to Swedish Application No. 2351313-8, filed Nov. 17, 2023, under 35 U.S.C. § 119 (a). Each of the above-referenced patent applications is incorporated by reference in its entirety.

### TECHNICAL FIELD

The present disclosure relates to monitoring of a common resource in a healthcare setting, and more specifically to methods and systems for determining a state of a central medical gas system.

### BACKGROUND

Healthcare settings often comprise various shared infrastructure resources, such as systems for power supply, climate control, wireless communication, and supply of medical gases. A central medical gas system is an example of such a resource, which is used in hospitals to deliver medical gases like oxygen, medical air, nitrous oxide, and other gases to patient rooms and operating theatres. The central medical gas system delivers the gases through a network of pipes directly to the actual location where healthcare services are rendered and may therefore replace individual gas cylinders or gas tanks. This frees up work surfaces and reduces the risk of strain injuries on healthcare staff as they no longer must handle heavy gas cylinders.

The performance of the central medical gas system is crucial for patient safety and effective medical treatment. Many medical procedures and treatments, such as surgeries and respiratory therapies, rely on a constant and uninterrupted supply of medical gases. Any interruption or inadequacy in supply could result in severe health consequences. For this reason, pressure gauges and flow meters may be installed at various points in the central medical gas system to monitor pressure and flow rates and ensure they are within specified ranges.

However, there is a need for improved and alternative technologies for monitoring the performance of common resources, such as central medical gas systems.

### SUMMARY

In view of the above, the present disclosure concerns improved or alternative methods and systems having the features set out in the independent claims.

Hence, according to a first aspect, there is provided a method for determining a state of a common resource, such as a central medical gas system. The method comprises receiving, from each of a plurality of medical devices in a healthcare setting, sensor data indicating a characteristic of a flow of a medical gas supplied to each of the plurality of medical devices from a common resource of the healthcare setting, and determining, based at least in part on the sensor data from each of the plurality of medical devices, a performance measure indicating a performance of the common resource. Thereafter, the performance measure is compared with a reference measure and the state of the common resource determined based at least in part on the comparison of the performance measure with the reference measure.

According to a second aspect, a system is provided, comprising a plurality of medical devices arranged in a healthcare setting and a common resource, such as a central medical gas system, arranged to supply each of the plurality of medical devices with a respective flow of medical gas. Each of the plurality of medical devices comprises a sensor configured to generate sensor data indicating a characteristic of the respective flow of medical gas. Further, the system comprises one or more processors, and one or more non-transitory computer-readable media storing computer executable instructions that, when executed by the one or more processors, cause the system to perform certain operations. The operations comprise, inter alia, receiving the sensor data, determining, based at least in part on the sensor data, a performance measure indicating a performance of the common resource, comparing the performance measure with a reference measure, and determining, based at least in part on the comparison, a state of the common resource.

A healthcare setting, such as a hospital, typically comprises several shared infrastructure resources, including systems for power supply, climate control, wireless communication, as well as supply and evacuation of medical gases. The shared resource typically supplies a plurality of individual nodes, such as point of care medical devices, vents, or radiators with a utility like electric power, medical gases, conditioned air, or wireless communication functionality. The utility may be provided by a central node and supplied to the individual nodes via distribution system. The central node may, for example, be a main grid connection, a backup generator, an air handling unit for filtering and tempering air, a central hub in a Wi-Fi network, or a central gas supply for medical gases. The utility may be distributed from the central node to individual or terminal nodes via distribution system such as an electric distribution system, a ductwork for conditioned air, a Wi-Fi network, or a medical gas piping system.

Some of these systems may comprise dedicated sensors for monitoring and supervision of the performance of the resource. The output from these sensors may be used as feedback in controlling the operation of the resource and issue a warning, should the performance fall outside acceptance ranges.

A central medical gas system is an example of such a shared, or common resource in which a medical gas may be distributed from a central supply to the actual location where healthcare services are rendered, such as a patient room or an operating theatre. The gas may be distributed to these locations through a network of pipes and outlets to a plurality of point of care medical devise delivering therapy to patients.

Each of these medical devices may comprise a sensor functionality for monitoring and controlling the performance of the medical device and ensure proper delivery of therapy to the patient. For this purpose, one or more sensors are typically built into the medical device. The sensor functionality may be arranged to measure flow rate and pressure of the medical gas supplied to an inlet of the medical device. The sensor functionality may thus be used to ensure proper delivery of therapy to the patient and allow a warning to be issued, should these characteristics of the gas supply fall outside acceptance ranges.

The inventors have realised that such a sensor functionality can be employed to monitor not only the performance of each individual medical device and characteristics of the flow of medical gas supplied to that device, but also to deduce information about the performance or state of the central medical gas system itself, such as the central gas supply and/or the distribution system carrying the medical gas from the central gas supply to the medical devices. The sensor data from the medical devices may be aggregated and analysed to determine a performance or state of the common resource without using sensors dedicated to the common resource (such as pressure sensors arranged at the central supply of the central medical gas system). In other words, sensor data that previously were used for monitoring the performance of individual medical devices may now be aggregated and used for the additional purpose of monitoring the common resource.

The term “healthcare setting” generally refers to a location where healthcare services are provided. Such a location may encompass a broad range of environments where medical care, diagnosis, treatment, or rehabilitation may occur. Typical examples of healthcare settings include hospitals, primary care clinics, specialised clinics, urgent care centres, ambulatory surgery centres, and long-term care facilities.

A “common resource” may be understood as a system that provides a utility to multiple nodes in the healthcare setting. In the above aspects, the common resource is a central medical gas system operable to deliver one or more medical gases to a plurality of point of care medical devices. The common resource may thus include a central gas supply and a distribution system for delivering the medical gas from the central gas supply to the medical devices. The central gas supply may comprise a single, centralised point of storage from which the utility is distributed to terminal nodes, or a plurality of points of storage from which the medical gas is made available to the medical devices. A healthcare setting may comprise two or more central medical gas systems that work in parallel to, for example, supply different types of medical gases to the point of care medical devices.

By a “characteristic” is generally meant a measurable property associated with the utility delivered by the common resource to an individual node. The characteristic may thus refer to flow rate, pressure, composition, or temperature of the medical gas supplied by the central medical gas system. The characteristic is typically indicated by the sensor data provided by each of the plurality of medical devices and may describe a measurable property of the flow of medical gas as experienced by an individual medical device.

The term “performance measure” may, in the context of the present disclosure, refer to a metric quantifying a property associated with the common resource. Typically, the performance measure relates to the central medical gas system's overall capability to provide the desired flow of medical gas, rather than the inlet flow received at each individual medical device. The performance measure may relate to a total flow rate, pressure, composition, or temperature of medical gas output from the central medical gas system, for example at a common access point. It is an underlying idea of the above-mentioned aspects to use sensor data from one or more of the individual medical devices to determine a state of the central medical gas system. By aggregating data from multiple individual nodes, a more comprehensive view of the central medical gas system's performance may be provided, thereby helping to identify trends, bottlenecks, or anomalies that may not be evident when looking at data from a single medical device or a sensor built into the central medical gas system. The sensor data hence allows for more accurate and reliable deductions to be made about the state of the central medical gas system. Sensor data from each of the medical devices can be aggregated when monitoring additive characteristics such as flow rates, whereas it may be sufficient to use sensor data from a single medical device when monitoring characteristics which generally are not additive. Examples of such characteristics include pressure of the gas delivered to a medical device, which typically is proportional to the flow rate and the flow resistance.

It will be appreciated that the term “performance” may refer to how well or efficiently the common resource provides a utility, whereas “performance measure” typically is a metric quantifying the performance.

As used in the present disclosure, the term “state” of the common resource generally refers to the condition or performance level of the common resource. In an operational or normal state, the common resource may deliver a utility within acceptance ranges. This state may be determined by comparing the performance measure of the common resource with a reference measure. In a degraded or malfunction state, the performance of the common resource may have dropped from its normal level. In this state, the medical devices may be incapable of delivering the intended therapy. In other examples, the medical device may still be functional, but at risk of not functioning properly. In other words, the patient safety may be compromised. Terms like “degraded”, “fault state”, “impaired”, or “need for service” may be used to describe this state.

As mentioned above, the state may be determined by comparing the performance measure with a reference measure. In case the performance measure lies outside an acceptance range, the state may be declared as “degraded” or “malfunctioning”. In some examples, the reference measure or acceptance range may be based at least in part on a previous performance value, which allows for fluctuations as well as drift over time to be determined.

In an example, a load distribution measure may be determined based on the sensor data. The load distribution measure may indicate the load distribution between the medical devices, and/or the load as a function of time. In other words, the medical gas consumed by each medical device may be analysed to detect potential bottlenecks and consumption peaks, and facilitate load balancing and more efficient resource utilisation.

In some examples, location data indicating a location of each of the medical devices may be used to determine the load distribution measure. The location data may for example include one or more of a bed, a room, a department, a floor, and a building of the healthcare setting. By providing the location data, the load placed on the common resource may be determined as a function of location within the healthcare setting. This allows for the medical gas consumption at each bed, room, department, floor, or building to be determined, which in turn may facilitate troubleshooting and redistribution of the medical gas supply within the healthcare setting.

In an example, the sensor data may be used to determine a total flow rate of the medical gas provided by the common resource. The total flow rate may then be compared with a reference flow rate to determine that there is a leakage. The reference flow rate may, for example, be a nominal flow rate of the medical gas output from the common resource or retrieved from measurements by a flow rate sensor arranged at an outlet of the common resource. A difference between the reference flow rate and the total flow rate, as calculated based on aggregated sensor data from each of the medical devices, and the reference flow rate may indicate there is a leakage along the path to the medical devices. Such an indication may be used to trigger a warning or increase the flow rate and/or pressure delivered by the common resource.

In some examples, the sensor data may indicate a local pressure at a medical device, i.e., the pressure of the flow of medical gas delivered to the medical device. The local pressure may be compared with a reference pressure to determine a performance, or state, of the common resource. The common resource may, for example, be determined to operate in a fault state, should the local pressure be lower than the reference pressure.

In a further example, the performance measure may be a pressure at the central supply, also referred to as a central pressure. The central pressure may be determined based on the local pressure delivered to a medical device, a flow rate at the medical device, and a predetermined flow resistance along the path between the central supply and the medical device. The flow resistance may be proportional to the driving pressure, i.e., the difference between the central pressure and the local pressure, and inversely proportional to the flow rate.

It will be appreciated that in further aspects of the present disclosure, sensor data from the plurality of devices may be employed to determine a state of common resources other than central medical gas systems, such as systems for power supply, wireless communication, and climate control.

Hence, in a third aspect, there is provided a method for determining a state of a common electric power supply. The method comprises receiving, from each of a plurality of medical devices in the healthcare setting, sensor data indicating a characteristic of electric power supplied to each of the plurality of medical devices from the common electric power supply of the healthcare setting, and determining, based at least in part on the sensor data from each of the plurality of medical devices, a performance measure indicating a performance of the common electric power supply. Thereafter, the performance measure is compared with a reference measure and the state of the common electric power supply is determined based at least in part on the comparison of the performance measure with the reference measure.

The characteristic may typically include voltage level, voltage balance (e.g. between the phases of a three-phase system), current level, and frequency of the electric power delivered to a medical device. The performance of the electric power supply may relate to the electric power supply's capability to delivering sufficient power, such as voltage and current, and its capability to handle peak loads, and potential load imbalances in the distribution system.

In a fourth aspect, there is provided a method for determining a state of a communication system, such as a local area network (LAN), a wireless local area network (WLAN), or a cellular network. The method comprises receiving, from each of a plurality of medical devices in the healthcare setting, sensor data indicating a characteristic of a communication service made available to each of the plurality of medical devices by the communication system, and determining, based at least in part on the sensor data from each of the plurality of medical devices, a performance measure indicating a performance of the communication system. Thereafter, the performance measure is compared with a reference measure and the state of the communication system is determined based at least in part on the comparison of the performance measure with the reference measure.

The characteristic of the communication service made available to each of the medical devices may typically include traffic load from each of the medical devices, signal strength, and latency experienced by the medical devices. This information can be used to determine a performance of the communication system, such as overall traffic load, throughput, bottlenecks, available bandwidth, and latency.

In a fifth aspect, there is provided a method for determining a state of a climate control system, such as an HVAC (Heating, Ventilation, and Air Conditioning) system in a healthcare setting. The method comprises receiving, from each of a plurality of medical devices in the healthcare setting, sensor data indicating a characteristic of an indoor environment in which the respective medical device is arranged. Further, the method comprises determining, based at least in part on the sensor data from each of the plurality of medical devices, a performance measure indicating a performance of the climate control system. Thereafter, the performance measure is compared with a reference measure and the state of the climate control system is determined based at least in part on the comparison of the performance measure with the reference measure.

The characteristic of the indoor environment may include temperature, humidity, air pressure, and air quality (such as levels of CO₂, particulates, and contaminants). The performance of the climate control system may relate to the climate control system's capability to deliver a required air flow with a certain temperature, humidity, or air quality.

Generally, the sensor data provided by each medical device and/or the determined state can be used as feedback for controlling or regulating the operation of the climate control system.

Each of the second to fifth aspects may generally have the same features and advantages as the first aspect. It is further noted that the disclosure relates to all possible combinations of features unless explicitly stated otherwise.

### BRIEF DESCRIPTION OF THE DRAWINGS

The above, as well as additional objects, features, and advantages of the present disclosure, will be better understood through the following illustrative and non-limiting detailed description of embodiments of the present disclosure, with reference to the appended drawings. Same reference numerals will be used for similar elements and acts.

FIG. 1 shows a plurality of medical devices connected to a central medical gas system according to an embodiment.

FIG. 2 shows a plurality of medical devices communicatively connected to a server according to an embodiment.

FIGS. 3a-c show flow charts of methods for determining a state of a common resource according to some embodiments.

FIG. 4 shows a plurality of medical devices connected to a common power supply according to an embodiment.

FIG. 5 shows a plurality of medical devices and a server on a communication network.

FIG. 6 shows a plurality of medical devices arranged in local environments controlled by a climate control system.

### DETAILED DESCRIPTION

Healthcare settings typically comprises various shared or common infrastructure resources in which a utility is distributed from a central node to a plurality of individual nodes by means of a distribution system. Examples of such utilities include medical gases, electric power, and local area network communication, which may be supplied to nodes including point of care medical devices such as, for example, devices for respiratory support and oxygenation support. The present disclosure relates to a technique for determining a state of the common resource, using sensor data from individual medical devices. By aggregating sensor data from each of the nodes, information about the performance of the common resource can be determined.

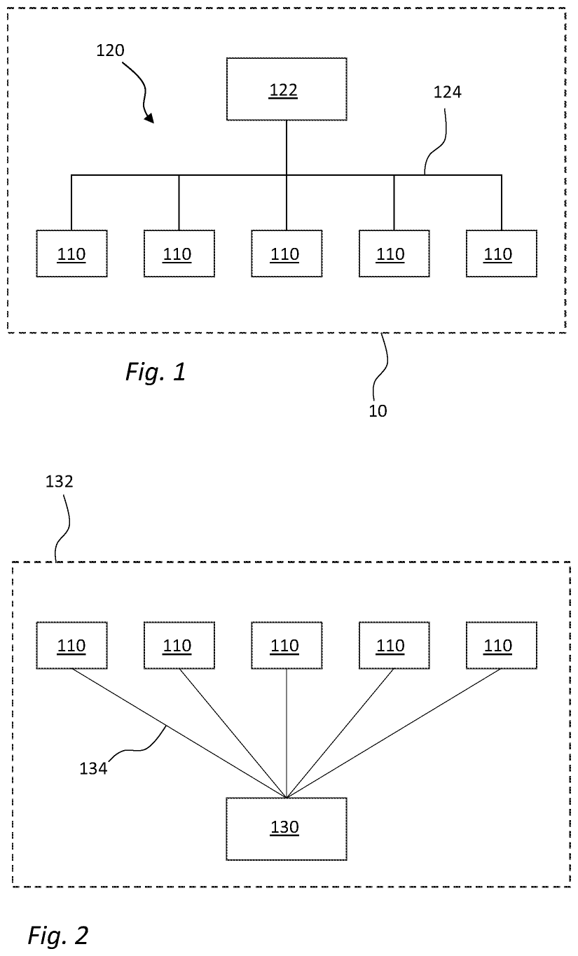

FIG. 1 shows by way of example a system 100 in a healthcare setting 10, in which a plurality of medical devices 110 are supplied with a utility from a common resource 120 via a distribution system 124. In the present example, the utility is a flow of medical gas, such as oxygen, medical air, or nitrous oxide, provided by a central medical gas supply 122 in a hospital 10.

The medical devices 110 may typically be point of care devices delivering therapy to a patient and may hence be arranged in a location where healthcare services are rendered. The location of each medical device 110 can be described by location data indicating, for example, a specific bed, a room, a department or ward, a floor, or a building of the healthcare setting. It will be appreciated that one or more medical devices 110 may share the same location, such that two or more of the medical devices 110 are providing therapy to the same patient, are arranged in the same room, or the same floor.

The medical devices 110 indicated in FIG. 1 may be devices for providing or assisting in patient respiratory support and/or oxygenation support. Examples include ventilators, heart-lung machines, and nebulisers administering drugs to the patient. The plurality of medical devices 110 may be of the same type or of two or more different types. Further, the common resource 120 may be arranged to provide the same type of medical gas, such as medical air, to each of the plurality of medical devices 110, or different types of medical gas to one or more of the medical devices 110.

The central medical gas system 120 comprises a central supply 122 (also referred to as a point of storage, or source equipment) including one or more bulk gas storage tanks for one or more medical gases, as well as compressors for medical air, and manifolds connecting multiple cylinders to ensure uninterrupted gas supply. The central supply 122 may be housed in a dedicated area, which typically may be purpose-built to accommodate the various components of the central supply 122. Further, a distribution system 124 or distribution network may be provided to carry the medical gas from the source 122 to various parts of the healthcare setting 10 and the individual nodes formed by the medical devices 110. The distribution system 124 comprises a network of piped lines as well as a valves and regulators for controlling pressure and flow rate.

In an example, the plurality of medical devices 110 comprises one or more ventilators 110, which is a type of medical devices 110 designed to assist or replace spontaneous breathing in patients who are unable to breathe adequately on their own. In invasive ventilation, a tube may be inserted into the patient's trachea through the mouth, whereas in non-invasive ventilation a mask covering the mouth of nose, or nasal masks or cannulas, can be used to assist breathing without the need for intubation. Ventilators 110 typically require careful monitoring to ensure that the patient is receiving the appropriate level of support. For this purpose, sensors and alarms may be provided to monitor conditions like inlet pressure and airway pressure, and to alert medical staff if dangerous conditions are detected. In the present example illustrated in FIG. 1, each medical device 110 comprises a sensor arrangement (not shown) configured to generate sensor data indicating pressure and/or flow rate of the medical gas delivered to the medical device 110 by the common resource 120. The sensor arrangement may be integrated in the medical device 110 and operable to measure characteristics such as pressure of the flow of medical gas at the inlet of the medical device 110.

FIG. 2 shows by way of example a plurality of medical devices 110, which may be similarly configured as the medical devices 110 shown in FIG. 1. The medical devices 110 are communicatively connected to a local server 130 according to an embodiment, comprising an environment for running an application that uses sensor data from the medical devices 110 to determine a state of the common resource 120. Each of the medical devices 110 and the local server 130 may be arranged on a local area network 132 of the healthcare setting 10. The medical devices 110 are thus communicatively connected to the local server 130 through a wired or wireless network connection 134. The local server 130 may comprise an application runtime environment, comprising a container management platform such as a local Kubernetes platform. The environment may also be referred to as a Control Centre and may form a platform for various applications providing real-time information to a user and assisting in analytic, reporting and maintenance of the connected devices 110 as well as the common resource 120.

During operation, each of the medical devices 110 may receive a respective flow of medical gas from the central medical gas system 120, as discussed above with reference to FIG. 1. One or more characteristics of the inlet flow of medical gas, i.e., the flow of medical gas received at each of the medical devices 110, may be indicated by sensor data sent to the local server 130 over the local network 132. The characteristics may, for example, include flow rate or pressure. The sensor data from each of the plurality of medical devices 110 may be processed by the local server 130 to determine a performance measure of the common resource 120. The performance measure may, for example, indicate a total flow rate delivered by the common resource 120, and more specifically a total flow rate of medical gas delivered from the central supply 122 to the distribution system 124. The total flow rate may be determined as a sum of the flow rate of medical gas delivered to each respective medical device 110. The total flow rate may then be compared with a reference rate, such as a predetermined nominal flow rate delivered by the central medical gas system 120 or a flow rate retrieved from a sensor arranged at an outlet of the central supply 122. In further examples, the performance measure may indicate a pressure, or central pressure, delivered by the central supply 122 to the distribution system 124. The difference in central pressure at the central supply 122 and the local pressure delivered to a medical device 110 may be referred to as the driving pressure, i.e., the pressure difference or pressure gradient driving the flow of medical gas from the central supply 122 to the medical device 110. The flow rate through the distribution system 124 may be quantified by Ohm's law of fluid flow, stipulating that the flow rate is proportional to the pressure gradient divided by the flow resistance along the flow path through the distribution system 124:

$$Q = \frac{{P}_{0} - {P}_{i}}{R}$$

where Q is the flow rate, P₀ is the pressure at the central supply, P₁ is the pressure at the medical device, and R is the flow resistance (which may be retrieved from previous measurements or considered to be a constant). Put differently, the central pressure P₀ at the central supply may be estimated as the pressure P₁ plus the pressure drop through the distribution system:

P₀=P+QRᵢ

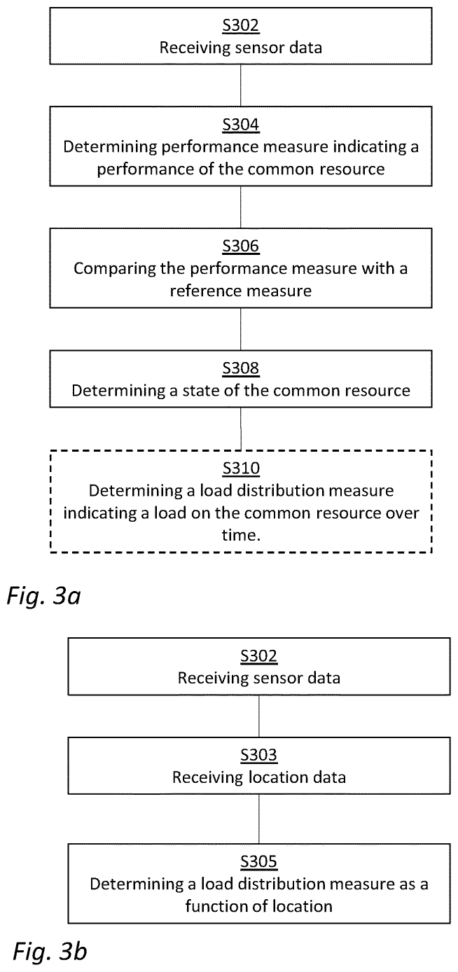

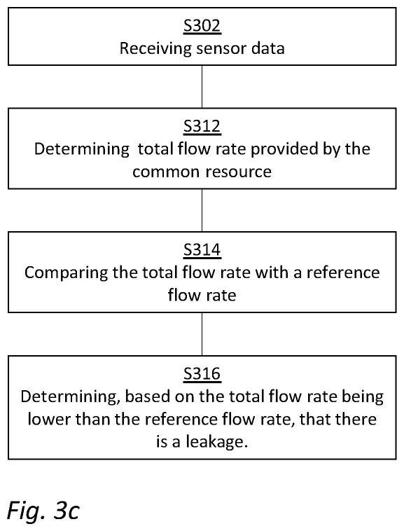

By comparing the total flow rate and/or the central pressure with reference values, a state of the central medical gas system 120 may be determined. Should the total flow rate obtained from the sensor data be lower than the flow rate at the outlet of the central supply 122, which may be retrieved from a sensor at the outlet, this may indicate there is a leakage along the path between the central supply 122 and any of the medical devices 110. Should no leakage be detected, a pressure drop detected at a medical device may indicate there is a bottleneck in the distribution system or that the overall capacity of the common resource is too low for the common resource to be able to deliver a gas flow at satisfactory flow rates and/or pressures. Similarly, an overload state may be determined in case the total flow rate meets or exceeds a reference rate indicating a maximum flow rate capacity of the central medical gas system 120 or in case the central pressure drops below a reference pressure. The sensor data provided by the medical devices 110 can thus be employed to determine an array of performance measures and states of the common resource 120, of which a few examples will be explored in the following with reference to FIGS. 3a-c.

FIG. 3a shows by way of example acts of a method performed in relation to a plurality of medical devices 110 receiving medical gas from a central medical gas system 120 as discussed above in connection with FIGS. 1 and 2.

The medical devices 110 comprises a sensor functionality that generates sensor data indicating a characteristic of the flow of medical gas received from the central medical gas system 120. The sensor data from each of the medical devices 110 are received S302 at the local server 130, where the data are processed to determine S304 a performance measure indicating a performance of the central medical gas system 120. The performance measure may typically be compared S306 with a reference measure to determine S308 a state of the central medical gas system 120.

Each of the medical devices 110 may be configured to provide data including both personal identifiable information (PII) and non-PII. PII may be referred to as clinical data typically including patient identification information such as name and ID number, as well as treatment information such as oxygen concentration, tidal volume, blood flow rate, physiological responses, and other types of data that on its own, or with other information, can be used to identify an individual. Non-PII, on the other hand, typically relates to information that cannot be used to identify an individual. Examples of such information include equipment data like make and model, software version, maintenance information, and aggregate statistics on device usage and the like.

Both PII and non-PII may be valuable to access from other devices than the medical device 110, such as a local server 120. Such a server may comprise an application runtime environment in which the data may be used for various applications providing real-time information to a user and assisting in analytics, reporting and maintenance of the medical devices 110 and common resources 120 supplying the medical devices 110 with a utility. For example, PII may typically be used by a healthcare provider to monitor, control, and optimise the healthcare delivered to the patient. Non-PII may typically be used for service and troubleshooting, remote support, asset management, cost control, etc. Non-PII is generally considered less sensitive from a patent integrity point of view and may not be subject to the same restrictions as PII.

In some embodiments, the sensor data may be considered as non-PII data and treated accordingly. Thus, the sensor data may either be sent to a server 130 on a local network 132, such as a local area network (LAN) 132 of a healthcare setting, or an external server which may be controlled by an entity different from the one controlling the medical devices 110.

The sensor data is indicative of a characteristic of the flow of medical gas delivered to a respective medical device 110. The characteristic may, for example, include flow rate, pressure, temperature, humidity, or composition of the medical gas.

The pressure and the flow rate of the medical gas are critical parameters for delivering the correct concentration of oxygen or other medical gases to the patient. The pressure and flow rate received by an individual medical device 110 is determined by the total capacity of the central medical gas system 120, and it is therefore of interest to determine the total load placed on the central medical gas system 120 by the medical devices 110 (i.e., the total gas consumption of the medical devices 110). This may, for example, be determined by summing S312 the flow rates measured at each of the medical devices 110 to retrieve a total flow rate provided by the central medical gas system 120 and compare S314 the total flow rate with a reference flow rate. The reference flow rate may be a predetermined nominal flow rate, or a flow rate obtained in previous measurements. In the latter case, a current total flow rate may be compared S314 with one or more historical flow rates to identify drifts and deviations that may indicate a fault or leakage of medical gas. In further examples, the total flow rate may be compared with a reference flow rate indicating a maximum gas delivery capacity of the distribution system 124 to detect potential bottlenecks in the distribution system 124. Such bottlenecks, or local overloading, may be addressed by connecting one or more of the medical devices 110 to a different branch or circuit of the distribution system 124. In further examples, a pressure of the medical gas delivered to a medical device 110 may be employed to determine a pressure of the medical gas delivered by a central supply 122. As discussed above, a pressure drop without any indication of a leakage may indicate there is a local bottleneck in the distribution system 124 supplying the medical device 110 with the gas.

Leakage may in some examples be determined by comparing a total amount of gas delivered to each of the medical devices 110 (for example retrieved by aggregating sensor data from the medical devise 110) with a total amount of medical gas delivered to the central supply 122. Oxygen, for example, may be delivered to the central supply 122 in liquid form. By converting the amount of liquid oxygen into an amount of gaseous oxygen (at pressures and temperatures similar to the ones at the medical devices 110), the total amount of oxygen delivered to the medical devices 110 may be compared with the total amount of oxygen initially available at the central supply 110. Should the total amount of oxygen delivered to the medical devices 110 be lower than the amount of oxygen initially available at the central supply 122, this may be an indication of a leakage somewhere along the path between the central supply 122 and the sensors of the medical devices 110.

In some examples, location data, indicating a physical location of each of the medical devices 110, may be received S303 at the server 130. The location data may, for example, indicate at which bed a medical device 110 is installed, or in which room, ward, or floor the medical device 110 is located. The location data may be employed to determine S305 a load distribution measure indicating the load placed on the central medical gas system 120 as a function of location. Put differently, the location data allows for the medical gas consumption at a specific bed, or in a specific room, ward, floor, or building to be determined. By knowing the gas consumption in specific locations, measures may be taken to address potential bottlenecks, shortages, leakages, and the like.

In some examples, the load distribution measure may be determined S310 to indicate a gas consumption as a function of time. This allows for peaks in the load to be detected and, possibly, predicted. By retrieving information on the load as a function of time, overloading of the central medical gas system 120 may be avoided by temporarily increasing the supply of medical gas at the central supply 122 or temporarily reducing the consumption at one or more of the medical devices 110.

In further examples, each medical device 110 may be configured to generate sensor data indicative of one or more of temperature, humidity, or composition of the flow of medical gas delivered to the medical device 110. Each of these characteristics may be used in addition to, or as an alternative to, the flow rate and pressure discussed above to determine a state of the common resource. The temperature may be used to indicate the central medical gas system's 120 capability to deliver medical gas within an acceptable temperature range, humidity range, or composition range.

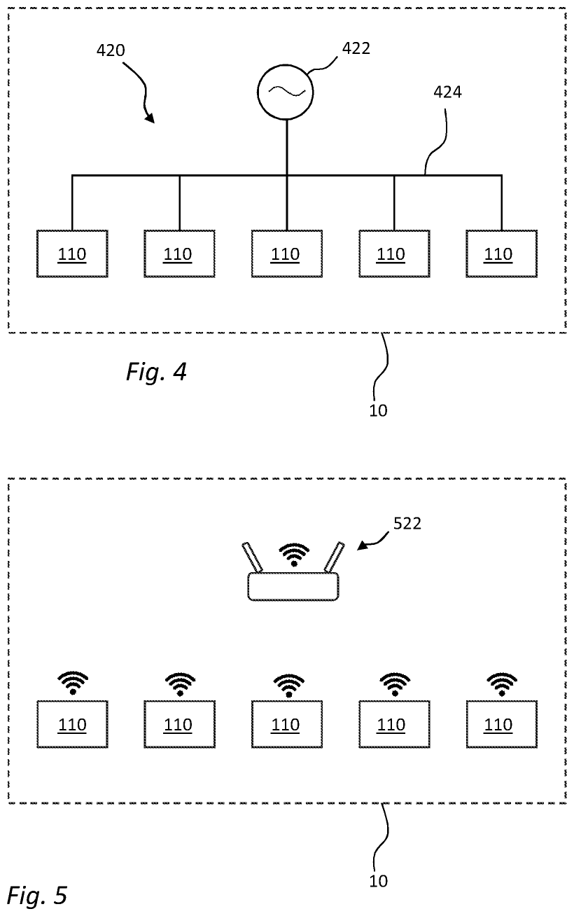

In an example, shown in FIG. 4, each of the medical devices 110 may be configured to generate sensor data indicating one or more properties of electric power supplied to the respective devices 110. The electric power may be provided by a common electric power supply 422, such as a main grid connection or a backup generator, which is connected to the plurality of medical devices 110 via a distribution network 424. The sensor data may indicate a characteristic of the electric power received at each medical device 110, including one or more of voltage, current, and phase balance. This data may be sent to the server 130 as discussed above in connection with FIG. 2, where the data may be processed to determine a performance of the common electric power supply 422. The performance of the common electric power supply 422 may, for example, be a metric quantifying the common electric power supply's 422 ability to deliver sufficient electric power of satisfactory quality to ensure proper operation of the plurality of medical devices 110. By comparing the performance measure with a reference measure, a state of the common electric power supply 422 may be determined. Should the common electric power supply 422, for example, be determined to operate in a state in which it is close to its maximum power capacity, a decision may be taken to connect a backup generator or energy storage, or to disconnect one or more of the medical devices and let them run on a battery backup instead.

In another example shown in FIG. 5, the medical devices 110 may generate sensor data indicating one or more properties of a communication service such as, for example, a local area network (LAN), a wireless local area network (WLAN), or a cellular network. The medical devices 110 shown in FIG. 5 are arranged on the same WLAN as the server 522, which may be similarly configured as the server 130 shown in FIG. 2. The sensor data may indicate a characteristic of the communication service, such as traffic load from the respective medical device 110 and signal strength. The sensor data may be processed by each of the medical devices 110 and/or a server 130 to determine a performance of the of the communication system, such as overall traffic load, throughput, potential bottlenecks between the medical devices 110 and the server 422, and available bandwidth in the communication system.

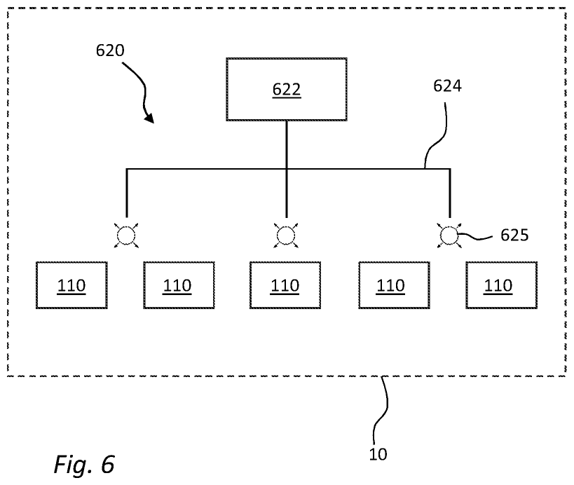

In yet a further example, depicted in FIG. 6, the plurality of medical devices 110 may generate sensor data that can be employed to determine a performance or a state of a climate control system 620, such as an HVAC (Heating, Ventilation, and Air Conditioning) system 620, controlling the indoor environment in which the medical devices 110 are located. Hence, one or more of the medical devices 110 may generate data characterising the indoor environment in the room or ward they are arranged. The HVAC system 620 indicated in FIG. 6 may comprise a central unit 622, such as an air handling unit where air is conditioned (heated, cooled, humidified, dehumidified, etcetera) before is it being sent through network of ducts 624 to vents and diffusers 625 in different locations of the healthcare setting.

The sensor data may indicate a characteristic of the local environment of the respective medical devices 110, such as temperature, humidity, air pressure, and air quality (such as levels of CO₂, particulates, and contaminants). The performance of the climate control system 620 may relate to its capability to deliver a required air flow with a certain temperature, humidity, or air quality to the various locations of the healthcare setting. The server 130 may be configured to process the sensor data to determine a performance measure indicating the performance of the climate control system 620, compare the measure with a reference measure, and determine a state of the climate control system 620 based on the sensor data from individual medical devices 110.

The medical devices 110 and/or the server 130 of the present disclosure may generally comprise one or more processors and one or more non-transitory computer-readable media storing first computer executable instructions that, when executed by the one or more processors, cause the medical device to perform at least parts of the actions shown in FIGS. 3a-c and described above.

Generally, the medical devices 110 and/or the server 130 may comprise circuitry which is configured to implement (using one or more non-transitory computer-readable media) the functionality described herein. Suitable processors for the execution of a program of instructions include, by way of example, both general and special purpose microprocessors, and the sole processor or one of multiple processors or cores, of any kind of computer. The processors can be supplemented by, or incorporated in, ASICs (application-specific integrated circuits). Those skilled in the art will understand that the above-described exemplary embodiments may be implemented in any suitable software, hardware, or firmware configuration or combination thereof. An exemplary hardware platform for implementing the exemplary embodiments may include, for example, an Intel x86 based platform with compatible operating system, a Windows OS, a Mac platform and MAC OS, a mobile device having an operating system such as iOS, Android, etc. In a further example, the exemplary embodiments of the above-described method may be embodied as a program containing lines of code stored on a non-transitory computer readable storage medium that, when compiled, may be executed on a processor or microprocessor.

Additionally, variations to the disclosed embodiments can be understood and effected by the skilled person in practising the claimed invention, from a study of the drawing, the disclosure, and the appended claims. Moreover, in the drawings and specification, there have been disclosed preferred embodiments and examples of the invention and, although specific terms are employed, they are used in a generic and descriptive sense only and not for the purpose of limitation. The scope of the invention is set forth in the following claims, in which the word ‘comprising’ does not exclude other elements or steps, and the indefinite article ‘a’ or ‘an’ does not exclude a plurality.

### CLAUSES

Clause 1. A method comprising:
   receiving (S302), from each of a plurality of medical devices (110) in a healthcare setting, sensor data indicating a characteristic of a flow of a medical gas supplied to each of the plurality of medical devices from a common resource (120) of the healthcare setting, the sensor data being generated by a respective sensor integrated into each of the plurality of medical devices;
   determining (S304), based at least in part on the sensor data from each of the plurality of medical devices, a performance measure indicating a performance of the common resource;
   comparing (S306) the performance measure with a reference measure; and
   determining (S308), based at least in part on the comparison of the performance measure with the reference measure, a state of the common resource.
Clause 2. The method according to clause 1, comprising:
   determining that the performance measure lies outside an acceptance range; and
   determining the state of the common resource based at least in part on the performance measure lying outside the acceptance range.
Clause 3. The method according to clause 2, wherein the acceptance range is based at least in part on a previous performance measure.
Clause 4. The method according to any of the preceding clauses, further comprising: determining, based at least in part on the sensor data, a load distribution measure indicating a load placed on the common resource over time.
Clause 5. The method according to clause 1, further comprising:
   receiving, from each of the plurality of medical devices, location data indicating a location of each of the medical devices, the location data indicating at least one of a bed, a room, a department, a floor, and a building of the healthcare setting.
Clause 6. The method according to clause 5, further comprising:
   determining, based at least in part on the sensor data and the location data, a load distribution measure indicating a load placed on the common resource as a function of location within the healthcare setting.
Clause 7. The method according to any of the preceding clauses, wherein the characteristic is selected from a list consisting of: pressure, flow rate, temperature, and composition.
Clause 8. The method according to any of clauses 1 to 6, wherein:
   the characteristic is a flow rate; and
   the performance measure indicates a total flow rate provided by the common resource.
Clause 9. The method according to clause 8, further comprising:
   determining the total flow rate of the medical gas based at least in part on the sensor data;
   comparing the total flow rate with a reference flow rate; and
   determining, based at least in part on the total flow rate being lower than the reference flow rate, that there is a leakage.
Clause 10. The method according to any of clauses 1 to 6, wherein:
   the characteristic is a local pressure at one or more of the plurality of medical devices;
   the method further comprising:
   comparing the local pressure with a reference pressure; and
   determining, based at least in part on the local pressure being lower than the reference pressure, that the common resource is operating in a fault state.
Clause 11. The method according to clause 10, wherein:
   the performance measure is a central pressure at a central supply (122) of the common resource (120);
   the method further comprising:
   determining the central pressure based on the local pressure, a flow rate at said at least one medical device, and a predetermined flow resistance between the central supply and said at least one medical device.
Clause 12. The method according to any of the preceding clauses, wherein each of the plurality of medical devices is a device for patient respiratory support or oxygenation support.
Clause 13. A system (100) comprising:
   a plurality of medical devices (110) configured to be arranged in a healthcare setting;
   a common resource (120) arranged to supply each of the plurality of medical devices with a respective flow of medical gas;
   wherein each of the plurality of medical devices comprises a respective sensor integrated into each of the plurality of medical devices and configured to generate sensor data indicating a characteristic of the respective flow of medical gas;
   the system further comprising:
   one or more processors; and
one or more non-transitory computer-readable media storing computer executable instructions that, when executed by the one or more processors, cause the system to perform operations comprising:
   receiving the sensor data (S302);
   determining (S304), based at least in part on the sensor data, a performance measure indicating a performance of the common resource;
   comparing (S306) the performance measure with a reference measure; and
   determining (S308), based at least in part on the comparison of the performance measure with the reference measure, a state of the common resource.
Clause 14. The system according to clause 13, wherein the common resource comprises a central supply (122) and a distribution system (124), wherein the distribution system is arranged to carry the medical gas from the central supply to each of the plurality of medical devices.
Clause 15. The system according to clauses 13 or 14, wherein the sensor is at least one of a pressure sensor and a flow rate sensor.

1. A method comprising:
receiving, from each of a plurality of medical devices in a healthcare setting, sensor data indicating a characteristic of a flow of a medical gas supplied to each of the plurality of medical devices from a common resource of the healthcare setting, the sensor data being generated by a respective sensor integrated into each of the plurality of medical devices, the sensor being configured to monitor a performance of the respective medical device;
determining, based at least in part on the sensor data from each of the plurality of medical devices, a performance measure indicating a performance of the common resource;
comparing the performance measure with a reference measure; and
determining, based at least in part on the comparison of the performance measure with the reference measure, a state of the common resource.

2. The method according to claim 1, comprising:
determining that the performance measure lies outside an acceptance range; and
determining the state of the common resource based at least in part on the performance measure lying outside the acceptance range.

3. The method according to claim 2, wherein the acceptance range is based at least in part on a previous performance measure.

4. The method according to claim 1, further comprising:
determining, based at least in part on the sensor data, a load distribution measure indicating a load placed on the common resource over time.

5. The method according to claim 1, further comprising:
receiving, from each of the plurality of medical devices, location data indicating a location of each of the medical devices, the location data indicating at least one of a bed, a room, a department, a floor, and a building of the healthcare setting.

6. The method according to claim 5, further comprising:
determining, based at least in part on the sensor data and the location data, a load distribution measure indicating a load placed on the common resource as a function of location within the healthcare setting.

7. The method according to claim 1, wherein the characteristic is selected from a list consisting of: pressure, flow rate, temperature, and composition.

8. The method according to claim 1, wherein:
the characteristic is a flow rate; and
the performance measure indicates a total flow rate provided by the common resource.

9. The method according to claim 8, further comprising:
determining the total flow rate of the medical gas based at least in part on the sensor data;
comparing the total flow rate with a reference flow rate; and
determining, based at least in part on the total flow rate being lower than the reference flow rate, that there is a leakage.

10. The method according to claim 1, wherein:
the characteristic is a local pressure at one or more of the plurality of medical devices;
the method further comprising:
comparing the local pressure with a reference pressure; and
determining, based at least in part on the local pressure being lower than the reference pressure, that the common resource is operating in a fault state.

11. The method according to claim 10, wherein:
the performance measure is a central pressure at a central supply of the common resource;
the method further comprising:
determining the central pressure based on the local pressure, a flow rate at said at least one medical device, and a predetermined flow resistance between the central supply and said at least one medical device.

12. The method according to claim 1, further comprising determining, based at least in part on the sensor data from each of the plurality of medical devices, a performance of the respective medical device.

13. The method according to claim 1, wherein the state of the common resource is determined without requiring additional sensors dedicated to the common resource.

14. The method according to claim 1, wherein each of the plurality of medical devices is a device for patient respiratory support or oxygenation support.

15. A system comprising:
a plurality of medical devices configured to be arranged in a healthcare setting;
a common resource arranged to supply each of the plurality of medical devices with a respective flow of medical gas;
wherein each of the plurality of medical devices comprises a respective sensor integrated into each of the plurality of medical devices, the sensor being configured to monitor a performance of the respective medical device and to generate sensor data indicating a characteristic of the respective flow of medical gas;
the system further comprising:
one or more processors; and
one or more non-transitory computer-readable media storing computer executable instructions that, when executed by the one or more processors, cause the system to perform operations comprising:
receiving the sensor data;
determining, based at least in part on the sensor data, a performance measure indicating a performance of the common resource;
comparing the performance measure with a reference measure; and
determining, based at least in part on the comparison of the performance measure with the reference measure, a state of the common resource.

16. The system according to claim 15, wherein the common resource comprises a central supply and a distribution system, wherein the distribution system is arranged to carry the medical gas from the central supply to each of the plurality of medical devices.

17. The system according to claim 15, wherein the sensor is at least one of a pressure sensor and a flow rate sensor.

18. The system according to claim 15, wherein the one or more non-transitory computer-readable media stores computer executable instructions that, when executed by the one or more processors, cause the system to perform operations comprising:
determining, based at least in part on the sensor data from each of the plurality of medical devices, a performance of the respective medical device.
